(19) United States
(12) Patent Application Publication
(10) Pub. No.: US 2025/0218066 A1
(43) Pub. Date: Jul. 3, 2025

(54) MOTION CORRECTION WITH LOCALLY LINEAR EMBEDDING FOR HELICAL PHOTON-COUNTING CT

(71) Applicant: RENSSELAER POLYTECHNIC INSTITUTE, Troy, NY (US)

(72) Inventors: Ge Wang, Loudonville, NY (US); Mengzhou Li, Troy, NY (US)

(73) Assignee: RENSSELAER POLYTECHNIC INSTITUTE, Troy, NY (US)

(21) Appl. No.: 18/850,619

(22) PCT Filed: Mar. 27, 2023
(86) PCT No.: PCT/US23/16359

§ 371 Date: Sep. 25, 2024

Related U.S. Application Data
(60) Provisional application No. 63/323,751, filed on Mar. 25, 2022.

(51) Int. Cl. G06T 11/00; G06T 7/11; G06T 7/246; G06T 7/80

(52) U.S. Cl. CPC G06T 11/005; G06T 7/11; G06T 7/251; G06T 7/80; G06T 2207/10081; G06T 2207/20012; G06T 2207/30016; G06T 2211/412

(57) ABSTRACT

A method of motion correction image reconstruction for photon-counting CT images includes scanning a subject via a photon-counting CT scanner device to obtain measured projection data; performing, via motion correction circuitry of the motion correction system, a LLE motion correction algorithm on the measured projection data to obtain motion correction data; generating, via reconstruction circuitry of the motion correction system, reconstructed image data from the motion correction data; and outputting corrected image data based, at least in part, on the reconstructed image data. A binary bad pixel mask is applied to exclude contributions from the bad pixels to the measured projection data. An unreliable volume mask is applied to exclude contributions from X-ray beams that passed through unreliable portions of the reconstructed image data. A virtual static object removal algorithm is performed to remove a static object from the measured projection data.

### CROSS REFERENCE TO RELATED APPLICATION(S)

This application claims the benefit of U.S. Provisional Application No. 63/323,751, filed Mar. 25, 2022, which is incorporated by reference as if disclosed herein in its entirety.

### GOVERNMENT LICENSE RIGHTS

This invention was made with government support under award number CA237267, awarded by the National Institutes of Health. The government has certain rights in the invention.

### FIELD

The present disclosure relates to motion correction with locally linear embedding, in particular to, motion correction with locally linear embedding for helical photon-counting computed tomography (CT).

### BACKGROUND

Motion-induced image artifacts are a long-standing problem in X-ray computed tomography (CT), which compromises many clinical CT scans. For instances, head CT and cardiac CT often suffer from image artifacts due to motions of either rigid structures (such as the head) or nonrigid tissues and organs (such as the heart). The rigid motion of an object can be equivalently treated as the view-dependent transformation of the locations of the source and detector pair. Without proper compensation for the motion, the reconstructed image quality will be degraded by the geometric errors, resulting in blurring, double-edging, and strong streaks.

X-ray photon-counting detector (PCD) technology, while advancing CT, may further contribute to challenges for motion estimation and correction. Due to the complex manufacturing process, PCDs may contain a significant number amount of ineffective pixels.

A large PCD array may contain a number of tiled small PCD chips. In the tiling process, gaps may be formed to, for example, provide adequate space for electronics. Both the gaps and the ineffective pixels are called bad pixels that can fail to record projection values. In some situations, the number of bad pixels may be so high that the missing values cannot be satisfactorily addressed using classic interpolation methods. As a result, it is difficult for traditional analytical reconstruction to be applied. Additionally, given a relatively high cost of a PCD compared to a flat panel detector of the same-area, a helical scan may be performed for an extended longitudinal field of view (FOV). Thus, motion estimation and correction may provide unique challenges.

### SUMMARY

In some embodiments, there is provided a method of motion correction image reconstruction for photon-counting computed tomography (CT) images. The method includes scanning a subject via a photon-counting CT scanner device to obtain measured projection data; transmitting the measured projection data to a motion correction system; performing, via motion correction circuitry of the motion correction system, a locally linear embedding (LLE) motion correction algorithm on the measured projection data to obtain motion correction data; generating, via reconstruction circuitry of the motion correction system, reconstructed image data from the motion correction data; and outputting corrected image data based, at least in part, on the reconstructed image data.

In some embodiments, the LLE motion correction algorithm includes estimating motion parameters for each of six degrees of freedom of the measured projection data to form six sub-problems; solving and updating the six sub-problems; and iterating the solving and updating until a convergence is reached. For each sub-problem the solving and updating includes generating a dense sample grid; calculating a reprojected projection grid; finding the K nearest neighbors from the projection grid in terms of Euclidean distance; optimizing the weights for the K neighbors; and updating the estimated motion parameters.

In some embodiments, for each iteration a sampling space for the sample grid is reduced while maintaining the same number of samples to generate a finer sample grid having improved searching accuracy.

In some embodiments, the six sub-problems are solved and updated sequentially. In other embodiments, the six sub-problems are solved and updated in parallel.

In some embodiments, the method further includes detecting, via bad pixel masking circuitry of the motion correction system, bad pixels of the photon-counting CT scanner; and applying a binary bad pixel mask to exclude contributions from the bad pixels to the measured projection data.

In some embodiments, the bad pixels are detected via open beam projection data and based, at least in part, on at least one detection criteria.

In some embodiments, the at least one detection criteria comprises a temporal mean of a pixel value is a statistical outlier in a group of all pixels. In some embodiments, the at least one detection criteria comprises a temporal variance of a pixel value is a statistical outlier in a group of all pixels. In some embodiments, the at least one detection criteria comprises a temporal mean of a pixel value is a statistical outlier in a group of all pixels, and a temporal variance of the pixel value is a statistical outlier in the group of all pixels.

In some embodiments, the photon-counting CT scanner is a helical photon-counting CT scanner, and the method further includes applying, via an unreliable volume masking circuitry of the motion correction system, an unreliable volume mask to exclude contributions from X-ray beams emitted by the photon-counting CT scanner that passed through unreliable portions of the reconstructed image data.

In some embodiments, applying the unreliable volume mask includes determining the unreliable portions of the reconstructed image data and generating a binary volume mask; forward projecting the binary volume mask to the measured projection data; and thresholding the projected results and generating the unreliable volume mask in the measured projection data.

In some embodiments, the unreliable portions are determined according to the Tam-Danielsson window. In other embodiments, the unreliable portions are determined by manually selecting a slice range per noise and image quality and reserving predetermined margins.

In some embodiments, the method further includes performing, via a virtual static object removal circuitry of the motion correction system, a virtual static object removal algorithm to remove a static object from the measured projection data.

In some embodiments, the virtual static object removal algorithm includes reconstructing an image volume on the measured projection data; segmenting a static object from the image volume with a predetermined margin and generating a static object mask; setting voxels outside of the static object mask to zero and reconstructing a static-object-only image volume; forward projecting the static-object-only image volume to obtain static-object-only projection data; and subtracting the static-object-only projection data from the measured projection data to obtain clean measured projected data for use in the LLE motion correction algorithm.

In some embodiments, the method further includes combining the static-object-only projection data with the reconstructed image data prior to generate the corrected image data.

In some embodiments, the static object is a support on which the subject is positioned during the scanning.

In some embodiments, there is provided a computer readable storage device. The device has stored thereon instructions that when executed by one or more processors result in the following operations including any embodiment of the method.

### BRIEF DESCRIPTION OF DRAWINGS

The drawings show embodiments of the disclosed subject matter for the purpose of illustrating features and advantages of the disclosed subject matter. However, it should be understood that the present application is not limited to the precise arrangements and instrumentalities shown in the drawings, wherein:

FIG. 1 illustrates an example projection with a 14-chip photon counting detector having a bad pixel mask applied according to embodiments of the present disclosure. The top portion shows the projection, and the bottom portion shows the bad pixel mask;

FIG. 2 illustrates example unreliable portions of a cone-beam geometry/helical CT scan. Any ray through an unreliable portion (shown at the top and bottom portions of the image volume) is unreliable and contributes to the mask indicating an unreliable area in a projection (shown at the top portion of the detector) which should be excluded in the loss calculation for motion estimation;

FIG. 3 illustrates a functional block diagram of a system that includes a motion correction system for motion correction for helical photon-counting computed tomography (CT), according to several embodiments of the present disclosure; and

FIG. 4 is a flowchart of operations for a method of motion correction for helical photon-counting CT, according to various embodiments of the present disclosure.

Although the following Detailed Description will proceed with reference being made to illustrative embodiments, many alternatives, modifications, and variations thereof will be apparent to those skilled in the art.

### DETAILED DESCRIPTION

Generally, this disclosure relates to motion correction with locally linear embedding for helical photon-counting computed tomography (CT). A method, apparatus and/or system may be configured to reduce or eliminate image artifacts that results from rigid-structure motion of the subject being imaged.

X-ray photon-counting detector (PCD) offers low noise, high resolution, and spectral characterization, representing a next generation of CT and enabling new biomedical applications. It is well known that involuntary patient motion may induce image artifacts with conventional CT scanning, and this problem becomes more serious with PCD due to its high detector pitch and extended scan time. Furthermore, PCD often comes with a substantial number of bad pixels, making analytic image reconstruction challenging and ruling out state-of-the-art motion correction methods that are based on analytical reconstruction. Embodiments of the present disclosure are directed to a locally linear embedding (LLE) correction method for the helical scanning geometry, which is especially desirable given the high cost of large-area PCD. Embodiments of the present disclosure also use an unreliable-volume mask to improve the motion estimation accuracy and perform incremental updating on gradually refined sampling grids for optimization of both accuracy and efficiency.

### LLE-Based Motion Correction

A rigid motion of a moving object is equivalent to a geometrical misalignment of the source and detector pair around a stationary object. The goal is to minimize the difference between measured projections and re-projected projections of an image volume reconstructed assuming estimated motion parameters. Mathematically, the optimization problem is formulated as follows:

$${arg min}_{p , x} {‖ b - A ( p ) x ‖}_{2}^{2} , \qquad ( 1 )$$

where A(p) is a system matrix after incorporating a vector motion parameters p, and x and b denote a reconstructed image and measured projections, respectively. The optimization may be performed by alternatively updating p and x. Since the motion parameters for one view is separable from those for other views, the problem can be dealt as a set of sub-problems for each view to update estimated motion parameters:

$$p = {[ {p}_{1} , … , {p}_{i} , … , {p}_{N} ]}^{T} , {p}_{i} = {arg min}_{{p}_{i}} {‖ {b}_{i} - {A}_{i} ( {p}_{i} ) x ‖}_{2}^{2} , \qquad ( 2 )$$

where i=1, 2, . . . , N denotes the ith view out of the N views in total, and pᵢ is the motion parameters for the ith view in the form of [tₓ, t_{y}, t_{z}, θₓ, θ_{y}, θ_{z}]^{T} characterized by six degrees of freedom, describing the translation and rotation along and around three Cartesian axes respectively. The minimum number of N depends on the resolution and the image noise. In some embodiments, N=180 for parallel beam reconstruction of a volume of 800×800×800. In some embodiments, N=3730 for a helical scan of a human subject's wrist with 10 rotations.

Different from gradient-based optimization, LLE utilizes a parallel searching strategy by densely sampling a pre-defined parametric range. The basic idea is that if the sampling grid is sufficiently small, the true vector of motion parameter should be so close to its K-nearest neighbors in the sampling grid and can be expressed as a linear combination of them such that its corresponding projection measurement can also be represented with the linear combination of the K-nearest reprojected projections associated with the sampled neighboring parameters and with the same weights, i.e.,

$${p}_{i}^{*} = {\sum }_{k = 1}^{K} {w}_{k} {p}_{i}^{( k )} , {b}_{i} = {\sum }_{k = 1}^{K} {w}_{k} {A}_{i} ( {p}_{i}^{( k )} ) x , \qquad ( 3 )$$

where Σ_{K=1}^{K}wₖ=1, and pᵢ^{(k)} is one of the K nearest samples for p*ᵢ on the grid (pᵢ⁽¹⁾, . . . , pᵢ^{(j)}, . . . , pᵢ^{(S)}) with S samples in total, and p*ᵢ denotes the ground truth motion parameters. This can be understood in light of the Taylor expansion of Aᵢ at p*ᵢ:

$${A}_{i} ( {p}_{i} ) \approx {A}_{i} ( {p}_{i}^{*} ) + {( {p}_{i} - {p}_{i}^{*} )}^{T} \frac{\partial {A}_{i} ( {p}_{i}^{*} )}{\partial {p}_{i}} , \qquad ( 4 )$$

$$Hence ,$$

$$\begin{matrix}{\sum }_{k = 1}^{K} {w}_{k} {A}_{i} ( {p}_{i}^{( k )} ) = {\sum }_{k = 1}^{K} {w}_{k} ( {A}_{i} ( {p}_{i}^{*} ) + {( {p}_{i}^{( k )} - {p}_{i}^{*} )}^{T} \frac{\partial {A}_{i} ( {p}_{i}^{*} )}{\partial {p}_{i}} ) \\ = {A}_{i} ( {p}_{i}^{*} ) + ( {\sum }_{k = 1}^{K} {{w}_{k} ( {p}_{i}^{( k )} - {p}_{i}^{*} )}^{T} ) \frac{\partial {A}_{i} ( {p}_{i}^{*} )}{\partial {p}_{i}}\end{matrix}$$

$$If {p}_{i}^{*} = {\sum }_{k = 1}^{K} {w}_{k} {p}_{i}^{( k )} , then :$$

$${\sum }_{k = 1}^{K} {w}_{k} {A}_{i} ( {p}_{i}^{( k )} ) = {A}_{i} ( {p}_{i}^{*} ) , {b}_{i} = {\sum }_{k = 1}^{K} {w}_{k} {A}_{i} ( {p}_{i}^{( k )} ) x . \qquad ( 5 )$$

In practice, sampling a multidimensional grid densely is too expensive. Instead, for each sub-problem embodiments of the present disclosure estimate the parameters of pᵢ individually and sequentially (six stages in total) as an effective approximation. All sub-problems can be simultaneously solved and updated for each stage. Specifically, for the rth parameter pᵢᵣ in one subproblem to be updated, the key steps are as follows:
1) Generate a dense sample grid {pᵢᵣ^{(s)}|s=1, . . . , S} for
2) Calculate the reprojected projection grid {(bᵢ^{(s)}, pᵢᵣ^{(s)}|s=1, . . . , S|} by replacing the rth parameter of pᵢ with the sampled values on the above grid;
3) Find the K nearest neighbors {(\tilde{b}ᵢ^{(k)}, \tilde{p}ᵢᵣ^{(k)}|k=1, . . . , K} for bᵢ from the projection grid in terms of the Euclidean distance;
4) Optimize the weights to w=[w₁, . . . , w_{K}]^{T} for the K neighbors to fit the measurement:

$$w = {arg min}_{w} ‖ {b}_{i} - {\sum }_{k = 1}^{K} {w}_{k} {\tilde{b}}_{i}^{( k )} ‖ s . t . {\sum }_{k = 1}^{K} {w}_{k} = 1 ; \qquad ( 6 )$$

5) Update the estimated parameter as pᵢᵣ=Σₖ₌₁^{K}wₖ\tilde{p}ᵢᵣ^{(k)}.
   The value of S depends on the extent of patient motion. Larger movement range needs larger S to ensure the sampling grid is dense in the unit of demagnified detector pixel size. For example, in some embodiments, 10 to 50 for a movement extent of [−5 mm, 5 mm] with 0.6 mm detector sampling size at the iso-center. In some embodiments, K=2 or 3 for computational efficiency.

Parallel computation can be implemented on GPU in a view-independent fashion for each degree of freedom, and the whole process can be iterated until a convergence is reached, similar to the process discussed in M. Chen, P. He, P. Feng, B. Liu, Q. Yang, B. Wei, and G. Wang, “General rigid motion correction for computed tomography imaging based on locally linear embedding,” Optical Engineering, vol. 57, no. 2, p. 023102, 2018, the contents of which are incorporated herein in its entirety.

### Bad Pixel Masking

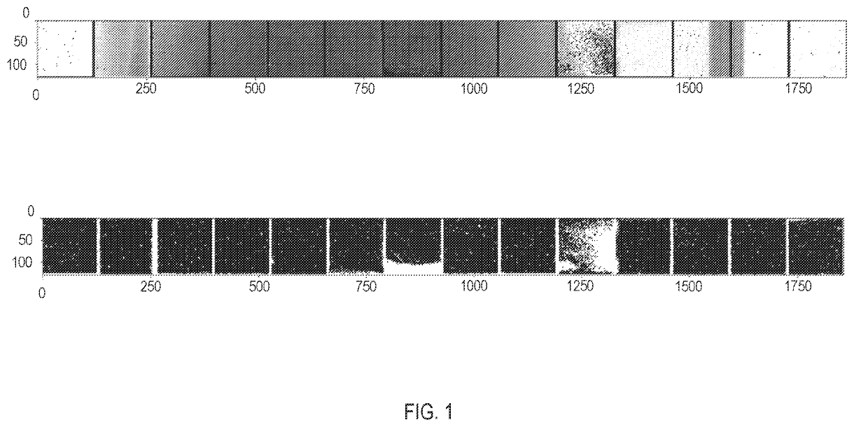

Most PCDs contain a substantial amount of bad pixels, including real ineffective pixels from the manufacturer, the tile gaps between chips, and dead/bad pixels due to degradation over time. These bad pixels produce unreliable responses and in a much larger number than that of a traditional energy-integrating detector. One exemplary projection with a 14-chip PCD is shown in FIG. 1. The top portion shows the projection, and the bottom portion shows the bad pixel mask. The number of bad pixels is so significant that a typical interpolation method is inadequate to address the problem without introducing major artifacts. Thus, bad pixel responses will directly reduce the motion estimation accuracy.

Embodiments of the present disclosure avoid the issue by turning off the bad pixels with a binary mask and utilize iterative reconstruction methods to avoid their contribution to the reconstruction and motion estimation. Bad pixels may be detected using, for example, open beam projection data, and based, at least in part, on one or more criteria. Detection criteria may include, but are not limited to, a temporal mean of a pixel value is a statistical outlier, in a group of all pixels, and/or a temporal variance of the pixel value is a statistical outlier, in a group of all pixels. The mask for the bad pixels will be used to exclude contributions from bad pixels during the volume reconstruction and in the calculation of the fidelity loss in Eq. 6 and facilitate motion estimation and image reconstruction. Regarding the term “statistical outlier” as used herein, embodiments of the present disclosure assume the good pixel values follow a Gaussian distribution with a mean value and a standard deviation sigma in an averaged open beam image (uniform X-ray source intensity). The pixels with deviation from the mean value greater than 3-sigma are treated as statistical outliers. The determination of the factor before sigma is depending on the perfectness of the detector and is a trade-off between stricter criteria for better pixel consistency and enough data points for stable reconstruction.

### Unreliable Volume Masking

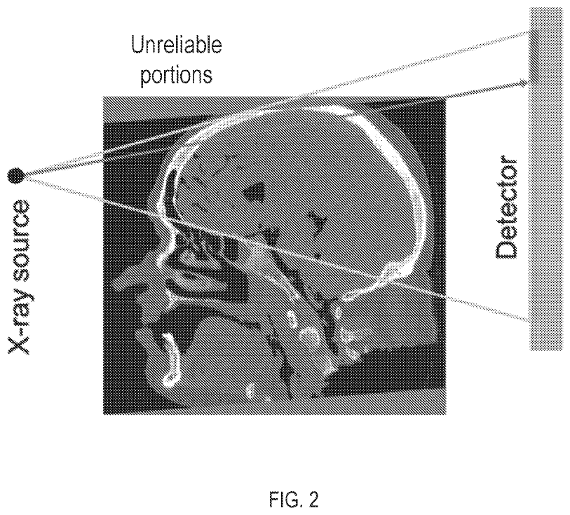

Due to the axial truncation in the cone-beam geometry/helical scan, there are unreliable portions at two longitudinal ends of a reconstructed volume due to data insufficiency, as shown in FIG. 2. Since the reconstruction of such a region is solely determined by a very limited angular range of projections resulting in a severe ill-posed problem, the strong limited angle artifacts will counter-balance the projection loss (Eq. 1) originated from misalignment and degrade the motion estimation accuracy.

To minimize this unreliability, embodiments of the present disclosure utilize a mask to exclude the contribution from the X-ray rays passing through the two unreliable portions of the reconstructed volume in computing the loss function in Eq. 6. This mask is generated in the following three operations:
1) Determine the unreliable portions and generate a volume mask (binary, zero for normal and one for unreliable voxels);
2) Forward project the volume mask to the projection domain; and
3) Threshold the projected results and generate the unreliable volume masks in the projection domain.

There are several different approaches to determine unreliable portions. For example, some embodiments determine reliability according to the Tam-Danielsson window, while other embodiments determine reliability by manually selecting the slice range per noise and image quality and reserving predetermined margins.

### Incremental Updating

Prior art motion correction algorithms directly estimate the motion vectors from an updated reconstruction in each iteration using the same dense sampling grid. However, the error of the motion estimation is expected to gradually decease as the algorithm iterates, which was observed in practice here. Global searching absolute values in a large grid may be computationally wasteful in later iterations. Hence, embodiments of the present disclosure use a more efficient local searching strategy on an incrementally refined grid for relative value; i.e., in each new iteration, the algorithm predicts correction to the motion estimation from the last iteration. Since the error is decreasing through iterations, the algorithm gradually shrinks the sampling space while maintaining the same number of samples to generate a finer grid for improved searching accuracy. Furthermore, embodiments use a much smaller number of samples compared to the absolute global searching to boost the searching efficiency without sacrificing performance.

### Virtual Bed Removal

In most CT scans, an object to be reconstructed is supported by a bed or a holder, which is static in reference to the scanning geometry. For example, a couch in a medical CT scanner or a sample holder in a micro-CT system. In the reconstructed image, the stationary bed or holder shows its sharp edges in a good image quality, regardless any object motion. However, during the motion correction process, those static structures are misaligned in reference to the object. In other words, after compensating for the motion of the object, the bed or holder will be blurred and degraded in the subsequently reconstructed image. This will prevent the loss function in Eq. 6 from being correctly minimized.

To avoid this undesired counterbalance effect, embodiments of the present disclosure virtually remove the static object (e.g., bed or holder) from projections, use the cleaned projections to perform motion correction, and then combined the reconstructed object with the previously reconstructed static object if needed. The operations for removal of the static object include:
1) Reconstruct an image volume in the initial scan geometry;
2) Segment the bed (i.e., all static portions) from the image volume with a proper margin and generate a bed mask;
3) Set voxels outside the bed mask to zero in the reconstruction for a bed-only image volume;
4) Forward project the bed-only volume to obtain the bed-only projections; and
5) Subtract the bed-only projections from the original projections to obtain the cleaned projections, with bed removed for motion estimation.

Thus, a system, according to the present disclosure, may be configured to reduce or eliminate image artifacts that results from rigid-structure motion of the subject being imaged by utilizing an LLE-based motion correction method for helical photon-counting CT, which decomposes the motion correction problem into each and every view with respective to individual parameters, and works iteratively in a highly parallel manner. Embodiments of the present disclosure exclude bad photon-counting detector pixels, and utilize unreliable volume masking, incremental updating, and incrementally refined gridding techniques synergistically. Thus, major improvements have been made in accuracy and efficiency of motion estimation and correction.

In one embodiment, there is provided an apparatus for motion correction for helical photon-counting CT. The apparatus is configured to perform a method of motion correction image reconstruction for photon-counting CT images, the method includes scanning a subject via a photon-counting CT scanner device to obtain measured projection data; transmitting the measured projection data to a motion correction system; performing, via motion correction circuitry of the motion correction system, a locally linear embedding (LLE) motion correction algorithm on the measured projection data to obtain motion correction data; generating, via reconstruction circuitry of the motion correction system, reconstructed image data from the motion correction data; and outputting corrected image data based on the reconstructed image data.

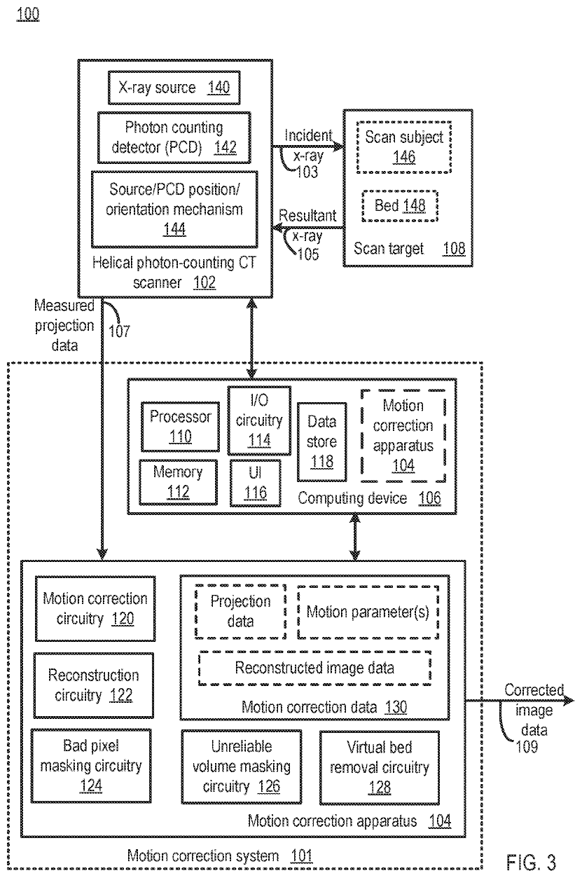

FIG. 3 illustrates a functional block diagram of a system 100 that includes a motion correction system 101 for motion correction for helical photon-counting CT, according to several embodiments of the present disclosure. System 100 further includes a helical photon-counting CT scanner 102 and a scan target 108. Helical photon-counting CT scanner 102 includes an X-ray source 140, a photon counting detector (PCD) 142, and a position/orientation mechanism 144 for the X-ray source 140 and the PCD 142. The scan target 108 includes a scan subject 146 and a support upon which the scan subject 146 is positioned, such as a bed 148. Motion correction system 101 includes a motion correction apparatus 104, and a computing device 106. Motion correction apparatus 104 may be coupled to or included in computing device 106. The motion correction system 101 is configured to reduce or eliminate image artifacts that results from rigid-structure motion of the subject being imaged, as will be described in more detail below.

Motion correction apparatus 104 includes motion correction circuitry 120, reconstruction circuitry 122, bad pixel masking circuitry 124, unreliable volume masking circuitry 126, and virtual bed removal circuitry 128. Motion correction circuitry 120 includes logic configured to receive the measured projection data 107 from the helical photon-counting CT scanner 102 and perform the LLE motion correction algorithm, as discussed above. The LLE motion correction algorithm includes estimating motion parameters for each of six degrees of freedom of the measured projection data to form six sub-problems, solving and updating the six sub-problems, and iterating the above solving and updating step until a convergence is reached. For each sub-problem the solving and updating includes generating a dense sample grid; calculating a reprojected projection grid; finding the K nearest neighbors from the projection grid in terms of Euclidean distance; optimizing the weights for the K neighbors; and updating the estimated motion parameters.

Reconstruction circuitry 122 includes logic configured to receive the motion correction data 130 outputted from the LLE motion correction algorithm and generate reconstructed image data, which is used to generate corrected image data 109 outputted from the motion correction system 101.

Bad pixel making circuitry 124 includes logic configured to detect bad pixels of the helical photon-counting CT scanner 102 and apply a binary bad pixel mark to exclude contributions from the bad pixels to the measured projection data 107, as discussed above.

Unreliable volume masking circuitry 126 includes logic configured to apply an unreliable volume mask to exclude contributions from X-ray rays emitted by the photon-counting CT scanner that passed through unreliable portions of the reconstructed image data, as discussed above. Applying the unreliable volume mask includes determining the unreliable portions of the reconstructed image data and generating a binary volume mask; forward projecting the binary volume mask to the measured projection data; and thresholding the projected results and generating the unreliable volume mask in the measured projection data.

Virtual bed removal circuitry 128 includes logic configured to perform the virtual static object (e.g., bed 148) removal algorithm to remove the bed from the measured projection data, as discussed above. The virtual static object removal algorithm includes reconstructing an image volume on the measured projection data; segmenting a static object from the image volume with a predetermined margin and generating a static object mask; setting voxels outside of the static object mask to zero and reconstructing a static-object-only image volume; forward projecting the static-object-only image volume to obtain static-object-only projection data; and subtracting the static-object-only projection data from the measured projection data to obtain clean measured projected data for use in the LLE motion correction algorithm. In some embodiments, the static-object-only projection data is combined with the reconstructed image data prior to generating the corrected image data.

Computing device 106 may include, but is not limited to, a computing system (e.g., a server, a workstation computer, a desktop computer, a laptop computer, a tablet computer, an ultraportable computer, an ultramobile computer, a netbook computer and/or a subnotebook computer, etc.), and/or a smart phone. Computing device 106 includes a processor 110, a memory 112, input/output (I/O) circuitry 114, a user interface (UI) 116, and data store 118.

Processor 110 is configured to perform operations of motion correction apparatus 104. Memory 112 may be configured to store data associated with motion correction apparatus 104. I/O circuitry 114 may be configured to provide wired and/or wireless communication functionality for system 100. For example, I/O circuitry 114 may be configured to receive measured correction data 107 and to provide corrected image data 109. UI 116 may include a user input device (e.g., keyboard, mouse, microphone, touch sensitive display, etc.) and/or a user output device, e.g., a display. Data store 118 may be configured to store one or more of measured correction data 107, corrected image data 109, and/or data associated with motion correction apparatus 104.

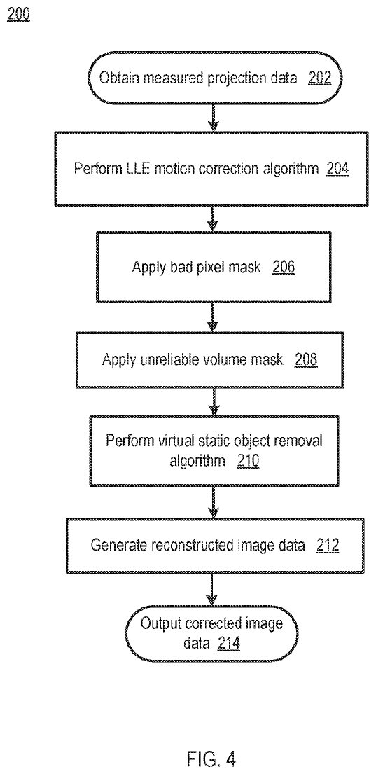

FIG. 4 is a flowchart 200 of operations for a method of motion correction for helical photon-counting CT, according to various embodiments of the present disclosure. In particular, the flowchart 300 illustrates a method of motion correction image reconstruction for photon-counting CT images. The operations may be performed, for example, by the system 100 of FIG. 3.

Operations of this embodiment may begin with scanning a subject via a photon-counting CT scanner device to obtain measured projection data at operation 202. Operation 204 includes performing, via motion correction circuitry of a motion correction system, a locally linear embedding (LLE) motion correction algorithm on the measured projection data to obtain motion correction data. Operation 206 includes detecting, via bad pixel masking circuitry of the motion correction system, bad pixels of the photon-counting CT scanner and applying a binary bad pixel mask to exclude contributions from the bad pixels to the measured projection data. Operation 208 includes applying, via an unreliable volume masking circuitry of the motion correction system, an unreliable volume mask to exclude contributions from X-ray beams emitted by the photon-counting CT scanner that passed through unreliable portions of the reconstructed image data. Operation 210 includes performing, via a virtual static object removal circuitry of the motion correction system, a virtual static object removal algorithm to remove a static object from the measured projection data. Operation 212 includes generating, via reconstruction circuitry of the motion correction system, reconstructed image data from the motion correction data. Operation 214 includes outputting corrected image data based, at least in part, on the reconstructed image data. Although the operations of motion correction method are shown in flowchart 200 in a certain order, this is not meant to limit operations of the motion correction method to that specific order. For example, embodiments of the present disclosure may perform operations 206, 208, and 210 in any order, whether before, after, or in parallel with operation 204.

Thus, a method, according to the present disclosure, may be configured to reduce or eliminate image artifacts that results from rigid-structure motion of the subject being imaged by utilizing an LLE-based motion correction method for helical photon-counting CT, which decomposes the motion correction problem into each and every view with respective to individual parameters, and works iteratively in a highly parallel manner. Embodiments of the present disclosure exclude bad photon-counting detector pixels, and utilize unreliable volume masking, incremental updating, and incrementally refined gridding techniques synergistically. Thus, major improvements have been made in accuracy and efficiency of motion estimation and correction.

As used in any embodiment herein, the terms “logic” and/or “module” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry,” as used in any embodiment herein, may include, for example, singly or in any combination, hardwired circuitry, programmable circuitry such as computer processors comprising one or more individual instruction processing cores, state machine circuitry, and/or firmware that stores instructions executed by programmable circuitry. The logic and/or module may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc.

Memory 112 may include one or more of the following types of memory: semiconductor firmware memory, programmable memory, non-volatile memory, read only memory, electrically programmable memory, random access memory, flash memory, magnetic disk memory, and/or optical disk memory. Either additionally or alternatively system memory may include other and/or later-developed types of computer-readable memory.

Embodiments of the operations described herein may be implemented in a computer-readable storage device having stored thereon instructions that when executed by one or more processors perform the methods. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

1. A method of motion correction image reconstruction for photon-counting computed tomography (CT) images, the method comprising:
scanning a subject via a photon-counting CT scanner device to obtain measured projection data;
transmitting the measured projection data to a motion correction system;
performing, via motion correction circuitry of the motion correction system, a locally linear embedding (LLE) motion correction algorithm on the measured projection data to obtain motion correction data;
generating, via reconstruction circuitry of the motion correction system, reconstructed image data from the motion correction data; and
outputting corrected image data based, at least in part, on the reconstructed image data.

2. The method of claim 1, wherein the LLE motion correction algorithm comprises:
estimating motion parameters for each of six degrees of freedom of the measured projection data to form six sub-problems;
solving and updating the six sub-problems, wherein for each sub-problem the solving and updating comprises:
generating a dense sample grid;
calculating a reprojected projection grid;
finding the K nearest neighbors from the projection grid in terms of Euclidean distance;
optimizing the weights for the K neighbors; and
updating the estimated motion parameters; and
iterating the above solving and updating step until a convergence is reached.

3. The method of claim 2, wherein for each iteration a sampling space for the sample grid is reduced while maintaining the same number of samples to generate a finer sample grid having improved searching accuracy.

4. The method of claim 2, wherein the six sub-problems are solved and updated sequentially.

5. The method of claim 2, wherein the six sub-problems are solved and updated in parallel.

6. method of any of the preceding claims, further comprising:
detecting, via bad pixel masking circuitry of the motion correction system, bad pixels of the photon-counting CT scanner; and
applying a binary bad pixel mask to exclude contributions from the bad pixels to the measured projection data.

7. The method of claim 6, wherein the bad pixels are detected via open beam projection data and based, at least in part, on at least one detection criteria.

8. The method of claim 7, wherein the at least one detection criteria comprises a temporal mean of a pixel value is a statistical outlier in a group of all pixels.

9. The method of claim 7, wherein the at least one detection criteria comprises a temporal variance of a pixel value is a statistical outlier in a group of all pixels.

10. The method of claim 7, wherein the at least one detection criteria comprises a temporal mean of a pixel value is a statistical outlier in a group of all pixels, and a temporal variance of the pixel value is a statistical outlier in the group of all pixels.

11. The method of any of the preceding claims, wherein the photon-counting CT scanner is a helical photon-counting CT scanner, and the method further comprising:
applying, via an unreliable volume masking circuitry of the motion correction system, an unreliable volume mask to exclude contributions from X-ray beams emitted by the photon-counting CT scanner that passed through unreliable portions of the reconstructed image data.

12. The method of claim 11, wherein applying the unreliable volume mask comprises:
determining the unreliable portions of the reconstructed image data and generating a binary volume mask;
forward projecting the binary volume mask to the measured projection data; and
thresholding the projected results and generating the unreliable volume mask in the measured projection data.

13. The method of claim 12, wherein the unreliable portions are determined according to the Tam-Danielsson window.

14. The method of claim 12, wherein the unreliable portions are determined by manually selecting a slice range per noise and image quality and reserving predetermined margins.

15. The method of any of the preceding claims, further comprising performing, via a virtual static object removal circuitry of the motion correction system, a virtual static object removal algorithm to remove a static object from the measured projection data.

16. The method of claim 15, wherein the virtual static object removal algorithm comprises:
reconstructing an image volume on the measured projection data;
segmenting a static object from the image volume with a predetermined margin and generating a static object mask;
setting voxels outside of the static object mask to zero and reconstructing a static-object-only image volume;
forward projecting the static-object-only image volume to obtain static-object-only projection data; and
subtracting the static-object-only projection data from the measured projection data to obtain clean measured projected data for use in the LLE motion correction algorithm.

17. The method of claim 16, further comprising combining the static-object-only projection data with the reconstructed image data prior to generate the corrected image data.

18. The method of claim 15, wherein the static object is a support on which the subject is positioned during the scanning.

19. A computer readable storage device having stored thereon instructions that when executed by one or more processors result in the following operations comprising the method according to claim 1.
